SEQUENCE LISTING (8 sequences)

SEQ ID NO: 1 - DNA, 17 bp, Artificial Sequence
  Primer
ccnctcaatgcctggag

SEQ ID NO: 2 - DNA, 22 bp, Artificial Sequence
  Primer
gcagtaccacaaggcctttcgc

SEQ ID NO: 3 - DNA, 19 bp, Artificial Sequence
  Primer
cgtctagccatggcgttag

SEQ ID NO: 4 - DNA, 19 bp, Artificial Sequence
  Primer
ggaactactgtcttcacgc

SEQ ID NO: 5 - DNA, 17 bp, Artificial Sequence
  Primer
acggtctacgagacctc

SEQ ID NO: 6 - DNA, 25 bp, Artificial Sequence
  Primer
ngcnacccaacnctactnggctagt

SEQ ID NO: 7 - DNA, 38 bp, Artificial Sequence
  Primer
naccttcaccctcagaaggngccgctcaatgcctggag

SEQ ID NO: 8 - DNA, 40 bp, Artificial Sequence
  Primer
naccttcaccctcagaaggngcgnctagccatggcgttag

(19) United States
(12) Patent Application Publication
(10) Pub. No.: US 2005/0164165 A1
(43) Pub. Date: Jul. 28, 2005

(54) Methods of detecting hcv genotype 1 (hcv-1) by using primers specific for the 5' non-coding region (ncr) of the hcv genome

(76) Inventor: William Malcolm Charles Rosenberg, Southampton (GB)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/501,262

(22) PCT Filed: Jan. 10, 2003
(86) PCT No.: PCT/GB03/00064

(30) Foreign Application Priority Data
Jan. 11, 2002 (GB) 0200526.2

(51) Int. Cl. C12Q 1/70; C12Q 1/68; C07H 21/04

(52) U.S. Cl. 435/5; 435/6; 536/23.72

(57) ABSTRACT

The present invention provides a method of detecting HCV genotype 1 (HCV-1) in a sample which is based on the finding that the 5′ non-coding region (NCR) of the HCV genome is conserved between HCV-1 quasi-species but not between other HCV subgroups. The method comprises subjecting the sample to an amplification reaction using at least one primer which anneals specifically to the 5′ noncoding region (5′ NCR) of the HCV-1 genome, and detecting the product of the amplification reaction. Kits and primers suitable for carrying out the method are also provided.

This invention relates to methods for detecting viral genotypes, and in particular Hepatitis C virus (HCV) genotypes. The invention therefore finds application in determining prognosis, and in the selection of treatment regimes, for patients infected with HCV.

The hepatitis C virus is a positively stranded RNA virus that exists in six genetically distinct genotypes. These are designated Type 1, 2, 3, 4, 5 and 6, and their full length genomes have been reported (Genbank/EMBL accession numbers Type 1a: M62321 (Choo), AF009606, AF011753, Type 1b: AF054250, D13558, L38318, U45476, D85516; Type 2b: D10988; Type 2c: D50409; Type 3a: AF046866; Type 3b: D49374; Type 4: WC-G6, WC-G11, WG29 (Li-Zhe Xu et al, J. Gen. Virol. 1994, 75: 2393-98), EG-21, EG-29, EG-33 (Simmonds et al, J. Gen. Virol. 1994, 74: 661-668)). Viruses in each genotype exist as differing “quasispecies” that exhibit minor genetic differences. In the UK and USA, the vast majority of infected individuals are infected with genotype 1, 2 or 3 HCV. In the UK, the only other genotype encountered is genotype 4 which is identified at low prevalence in some immigrant populations. HCV infection affects approximately 0.5% of the population in the United Kingdom, 1.8% of the population in the USA and 3% of the population of the world. In over 85% of infected people, HCV causes a lifelong infection characterised by chronic hepatitis that varies in severity between individuals.

The only effective therapy that is currently available is interferon alpha (IFN-α) used alone or in conjunction with ribavirin (“combination therapy”). Randomised controlled trials have shown that combination therapy is highly effective in the treatment of HCV infection in patients previously treated with interferon alone and in patients never previously treated with interferon (Davis et al, NEJM, (1998), 339(21): 1493-99; Poynard et al, Lancet (1998) 352(9138): 1426-32).

Subgroup analysis has revealed that the major determinant of prognosis and response to treatment is HCV genotype (Poynard et al, Lancet, 1998, 352(9138): 1426-32). For patients with genotype 2 or 3 infections treated for six months, response rates to treatment for six months are equivalent to those seen in patients with genotype 1 infections for one year. There were insufficient data in the trials on response rates for patients with genotype 4, 5 and 6 infections to permit investigators to determine response rates in patients with these infections. In mixed genotype infections, genotype 1 predominates.

Combination therapy is expensive (in the UK, it costs approximately £8,000 for one year's treatment). Thus, the identification of patients with non-genotype 1 infections allows treatment to be stopped after 6 months with a consequent cost saving, and the avoidance of unnecessary adverse effects of the drugs associated with further treatment. In addition, the most serious outcome of HCV infection is heptocellular carcinoma. It is therefore desirable to be able to identify with which genotype(s) of HCV a patient is infected.

Known HCV genotyping methods rely on agarose gel electrophoresis of restriction endonuclease digested polymerase chain reaction amplification products (RFLP) or antibody detection of anti-HCV antibodies. Enzymatic digestion can take a relatively long time to reach completion making the RFLP method time consuming. It also involves several different stages, making it unamenable to automation or rapid throughput. In addition, inaccurate genotyping can occur owing to (i) the fact that restriction endonucleases do not cut with 100% efficiency, and (ii) single base pair mutations in quasispecies may result in failure of digestion of the PCR products. Antibody based methods are costly and relatively insensitive. Most significantly, they are incapable of differentiating between patients who have cleared HCV infection and those with persisting infection. They thus require the performance of a virological test in parallel to confirm current infection.

U.S. Pat. No. 5,851,759 discloses methods of genotyping HCV in which HCV RNA is isolated and cDNA is synthesised from this RNA. The cDNA is then subjected to PCR using primers which flank the E1 region of the HCV genome. The products of PCR are analysed using the Heteroduplex Traching Assay (HTA). The E1 region was selected for analysis because it is considered to be the most heterogeneous region of the HCV genome. Thus, amplification of this region is likely to give a different product for each HCV strain in a sample. These products can then be analysed to indicate the strains that are present in the sample.

In view of the different effect of combination therapy on infection with HCV 1 as compared to infection with other HCV subtypes, there exists a need to be able to detect HCV genotype 1.

According to the present invention, there is provided a method of detecting HCV genotype 1 (HCV-1) in a sample, comprising:
subjecting the sample to an amplification reaction using at least one primer which anneals specifically to the 5′ noncoding region (5′ NCR) of the HCV-1 genome; and;
detecting the product of the amplification reaction.

The HCV genome consists of 5′ and 3′ noncoding (or untranslated) regions (NCR) that flank a single long open reading frame (ORF). This ORF encodes for three structural proteins at the amino-terminal end and for six non-structural (NS) proteins at the carboxyl-terminal end. The structural proteins are represented from the nucleocapsid (core; C) proteins and two glycoproteins, envelope 1 (E1) and envelope 2 (E2). The non-structural proteins are named NS2, P7, NS3, NS4a, NS4b, NS5a, NS5b. The 5′ NCR is the most highly conserved part of the HCV genome, whereas the sequence of the two envelope proteins (E1 and E2) is highly variable among different HCV isolates. The present invention lies in the surprising finding that parts of the 5′ NCR are conserved between HCV-1 quasi species but not between other genotypes. A preferred region is the region between residues −134 and −118 of the 5′NCR of HCV-1. Accordingly, primers for amplification reactions can be designed which anneal specifically to those parts of the 5′ NCR that are conserved between HCV-1 quasi species but not between other genotypes.

The amplification reaction may be PCR (see for example U.S. Pat. Nos. 4,683,195 and 4,683,202, and Innis et al, editors, PCR Protocols, (Academic Press, New York, 1989; Sambrook et al, Molecular Cloning, Second Edition, (Cold Spring Harbour Laboratory, New York 1989)). PCR will generally be used when HCV RNA has been isolated and converted, preferably by reverse transcription, to cDNA. Preferably, PCR is carried out using Taq DNA polymerase, e.g. Amplitaq™ (Perkin-Elmer, Norwalk, Conn.). Taq polymerase can also be obtained from MBI Fermentas, Perkin Elmer, Boehringer Mannheim and Beckman Instruments. An equivalent, preferably thermostable, DNA polymerase may also be used in the method of the present invention, such as Tfl (Thermus flavus) polymerase (Gut et al, Virol. Methods 77(1): 3746 (1999)).

Alternatively, the amplification reaction may be RT-PCR (Yajima et al, Clin. Chem, 44(12): 2441-2445 (1998); Martell et al, J. Clin. Microbiol., 37(2): 327-332 (1999); Gut et al, Virol. Methods 77(1): 37-46 (1999); Predhomme et al, Leukemia, 13(6): 957-964 (1999)), in which the HCV RNA is reverse transcribed into cDNA which is then subjected to PCR amplification.

As is well-known, PCR involves the extraction and denaturation (preferably by heat) of a sample of DNA (or RNA). A molar excess of oligonucleotide primers is added, along with a polymerase, which may be heat-stable, and dNTPs for forming the amplified sequence. The oligonucleotide primers are designed to hybridise to opposite ends of the sequence which it is desired be amplified. In the first amplification round, the polymerase replicates the DNA to produce two “long products,” which begin with the respective primers. The total DNA, which includes the two long products and the two original strands, is then denatured and a second round of polymerisation is carried out (for example, by lowering the temperature). The result of the second round is the two original strands, the two long products from the first round, two new long products (produced from the original strands), and two “short products” produced from the long products. These short products have the sequence of the target sequence (sense or antisense) with a primer at each end. For each additional amplification round, the number of short products grows exponentially, each round producing two additional long products and a number of short products equal to the sum of the long and short products remaining at the end of the previous round.

Oligonucleotide primers can be synthesised by a number of approaches, e.g. Ozaki et al, Nuc. Acids Res. 20: 5205-5214 (1992); Agrawal et al, Nuc. Acids Res. 18: 5419-5423 (1990) or the like. Conveniently, the oligonucleotide probes are synthesised on an automated DNA synthesiser, e.g. an Applied Biosystems, Inc, Foster City, Calif. model 392 or 394 DNA/RNA synthesiser using standard chemistries such as phosphoramidite chemistry (Beaucage and Iyer, Tetrahedron 48: 2223-2311 (1992), U.S. Pat. Nos. 4,980,460, 4,725,677, 4,415,732, 4,458,066 and 4,973,679). Alternative chemistries, including non-natural backbone groups such as phosphorothioate and phosphoramidate, may also be employed, provided that the hybridisation efficiencies of the resulting oligonucleotides are not adversely affected. The precise length and sequence of the DNA primers will depend on the target polynucleotide to be amplified Preferably, the length of the DNA primers is in the range 10 to 60 nucleotides and more preferably in the range 15 to 30 or 25 nucleotides.

In a preferred embodiment, the method of the present invention further comprises a step in which HCV infection generally is detected. In this step, the sample is subjected to an amplification reaction using primers which anneal to a region of the 5′NCR which is conserved between all HCV genotypes and the product of this amplification reaction is detected. Preferably, the amplified region spans the region which is conserved in HCV-1. This additional step allows verification that HCV is present in the sample and so treatment can be tailored accordingly if no HCV-1 is detected.

Detection of the products of amplification may be carried out using the well-known technique of agarose gel electrophoresis. If HCV-1 is present in the sample, the amplification reaction produces a product (of known size) which can be detected on the agarose gel. An HCV-1 specific primer suitable for this is the DNA oligonucleotide:

| | |
|---|---|
| 5′ CCI CTC AAT GCC TGG AG 3′ | (Spec-1) |

Spec-1 may be used together with any universal HCV primer. One example is:

| | |
|---|---|
| Reverse: | |
| 5′ GCA GTA CCA CAA GGC CTT TCG C 3′ | (UTR-R2) |

The invention provides such primers alone and in combination.

If it is desired to carry out an amplification reaction additionally to detect whether HCV in general is present in the sample, a pair of primers universal for all HCV genotypes can be used. This will produce a product of a known size which is different to that for the HCV-1-specific amplification. If HCV-1 and other HCV genotypes are present, the gel will show both products. Suitable primers for this are:

| | | |
|---|---|---|
| Forward: | 5′ CGT CTA GCC ATG GCG TTA G 3′ | (UTR-L2) |
| Reverse: | (UTR-R2) | |

Additionally, a preliminary amplification may be carried out on the sample to isolate HCV material. This preliminary amplification can be carried out using two primers universal for all HCV genotypes. Suitable primers for this are:

| | | |
|---|---|---|
| Forward: | 5′ GGA ACT ACT GTC TTC ACG C 3′ | (UTR-L1) |
| Reverse: | 5′ ACG GTC TAC GAG ACC TC 3′ | (UTR-R1) |

Detection of the products of amplification may alternatively be carried out using fluorescent analysis. If genotype 1 HCV is present, amplification occurs and this is detected by hybridisation of a quenched fluorescently labelled probe. With successive rounds of amplification, an increasing amount of the fluorescent probe becomes incorporated into PCR products and the quencher is liberated resulting in an increasing amount of fluorescence being detectable on the reaction vessels. Such fluorescence detection makes this approach ideally suited to automation and high throughput processes.

One well-known fluorescent technique is the so-called “TaqMan” method (Holland, et al, Proc. Nat. Acad. Sci, 88: 7276-80, (1985)). This method uses an oligonucleotide probe complementary to a segment of the target polynucleotide, i.e. between the sequence defined by the PCR primers, which probe is labelled with two fluorescent molecules or fluorophores. The emission spectrum of one of the molecules overlaps with the excitation spectrum of the other and, as a result, the emission of the first fluorophore (the “reporter”) is quenched by the second (the “quencher”) when they are in sufficient proximity.

The oligonucleotide probe is hybridised to the target polynucleotide downstream from a primer for a polymerase having a 5′-3′ exonuclease activity. As the polymerase extends the primer as part of the PCR reaction, the oligonucleotide is digested, releasing one of the “quencher” and “reporter” molecules and causing the distance between these molecules to become such that the fluorescent emission is no longer quenched by energy transfer. Thus, a fluorescent signal is generated, providing real-time monitoring of amplification.

The oligonucleotide probe may be made in the same way as the oligonucleotide primers mentioned above and may have the same variations in backbone and so on, providing that hybridisation to the target polynucleotide is not compromised and/or cleavage efficiency of the exonuclease are not adversely affected. Preferably, the length of the oligonucleotide probe is in the range of 10 to 60 nucleotides, and more preferably 18 to 30 nucleotides. The precise length and sequence of the oligonucleotide probe depends at least in part on the nature of the target polynucleotide to which it binds and will be varied to achieve appropriate annealing and melting properties for a particular target polynucleotide. In addition, the binding location of the probe may be varied according to the nature of the target polynucleotide.

Preferred primers for this reaction are UTR-R2 and Spec1 mentioned above. A preferred probe has the following sequence and is specific to HCV-1:

| | |
|---|---|
| 5′ FCG CIA CCC AAC ICT ACT IGG CTA GT 3′ | (L1) |

where F = 6-FAM, 3′-T + TAMRA

Alternatively, the technique such as the TaqMan technique can be used to detect HCV-1 using one or more universal primers and an HCV-1 specific probe. Thus, in a further aspect, the present invention provides a method of detecting HCV genotype 1 (HCV-1) in a sample, comprising:
subjecting the sample to an amplification reaction using at least one primer which anneals to the genome of HCV, a polymerase having a 5′-3′ exonuclease activity and an oligonucleotide probe, which probe anneals specifically to the 5′ noncoding region (5′ NCR) of the HCV-1 genome and which incorporates a modified nucleotide having a fluorescent characteristic which is modified by one or more neighbouring nucleotides; and
detecting a change in fluorescence as the oligonucleotide probe is degraded by the exonuclease activity of the polymerase as the polymerase extends the primer and modification of the fluorescent characteristic of the modified nucleotide is reduced.

The modified nucleotide is preferably a “reporter” molecule and the neighbouring nucleotide(s) is/are preferably a “quencher” molecule. The primer(s) may be universal and is/are preferably UTR-L1 and UTR-R1 or UTR-L2 and UTR-R2. Alternatively, the probe(s) may be capable of annealing specifically to the 5′ NCR of the HCV-1 genome. The probe may be L1 as defined above.

A further alternative method of detecting the amplification products is using one or more molecular beacon probes. These are PCR primers comprising a sequence attached to a hairpin loop sequence which contains both a fluorescent label and a quencher molecule. The hairpin brings the fluorescent molecule in opposition to the quencher so that the primer does not fluoresce under normal conditions. As PCR results in copying of the probe sequence, the hair-pin opens and the fluorescent beacon and quencher become separated. The resulting PCR product fluoresces.

In a preferred embodiment, the following PCR primers are used:

Forward: MBP-LR-1
5′FCA CCT TCA CCC TCA GAA GGM GCC GCT CAA TGC CTG
GAG3′
(F = FAM; M = MeREDdU and U = Uracil)
Reverse: UTR-R2

MBP-LR-1 is a forward HCV type 1 specific primer and, as mentioned above, UTR-R2 is a universal reverse primer. Any universal HCV primer can be used together with MBP-LR-1.

If it is desired to detect the presence of other HCV genotypes in the sample, an additional amplification can be carried out using at least one molecular beacon probe which is universal for all HCV genotypes. A suitable probe in this regard is MBP-LR-ALL which is a forward primer and has the following sequence:

5′FCACCTTCACCCTCAGAAGGMGCGUCTAGCCATGGCGTTAG3′
(F = FAM; M = MeREDdU and U = Uracil)

MBP-LR-ALL can be used together with any universal reverse primer; one suitable example is UTR-R2.

Fluorochromes that emit light at differing wavelengths can be incorporated into the different primers so that products amplified with different primers can be distinguished in the same reaction vessel. Alternative hairpin sequences on the beacon probes can easily be designed by the skilled person.

Alternatively, the amplification products can be detected using DNA hybridisation employing enzyme linked methods to confirm hybridisation, such as using horse radish peroxidase on sequence specific probes and an appropriate substrate.

According to a further aspect of the present invention, there is provided a kit for detecting HCV genotype 1 (HCV-1) in a sample, comprising: at least one primer which anneals specifically to the 5′ noncoding region (5′ NCR) of the HCV-1 genome.

The kit may further comprise the polymerase and an appropriate primer or set of primers. In addition, it may comprise additional reagents that are necessary for performing the invention, such as dNTP mixtures, buffers, molecular size standards, wax beads and the like. The reagents may be provided in pre-measured amounts so as to simplify the performance of the method. Typically, the kit may also contain detailed instructions for carrying out the method.

It will be appreciated by those skilled in the art that the sequences of the primers and probes described above can be modified without affecting their activity, i.e. their ability to act as primers or probes in the methods of the present invention. Such modified probes and primers are included in the present invention, provided that they have substantial identity with the probes mentioned above.

When comparing nucleic acid sequences for the purposes of determining the degree of homology or identity, one can use programs such as BESTFIT and GAP (both from the Wisconsin Genetics Computer Group (GCG) software package). BESTFIT, for example; compares two sequences and produces an optimal alignment of the most similar segments. GAP enables-sequences to be aligned along their whole length and finds the optimal alignment by inserting spaces in either sequence as appropriate. Suitably, in the context of the present invention when discussing identity of nucleic acid sequences, the comparison is made by alignment of the sequences along their whole length.

Preferably, sequences which have substantial identity have at least 75% sequence identity and more preferably at least 90 or at least 95% sequence identity with said sequences. In some cases, the sequence identity may be 99% or above.

Desirably, the term “substantial identity” indicates that said sequence has a greater degree of identity with any of the sequences described herein than with prior art nucleic acid sequences.

Preferred features of each aspect of the invention are as for each of the other aspects mutatis mutandis. The prior art documents mentioned herein are incorporated to the fullest extent permitted by law. The abbreviations for nucleotides used herein are standard unless otherwise indicated.

The present inventors have invented a polymerase chain reaction amplification (PCR) based test that permits the detection of all types of HCV infection and the specific identification of HCV genotype 1 infections.

The present invention will now be described further in the following examples. Reference is made to the accompanying drawings:

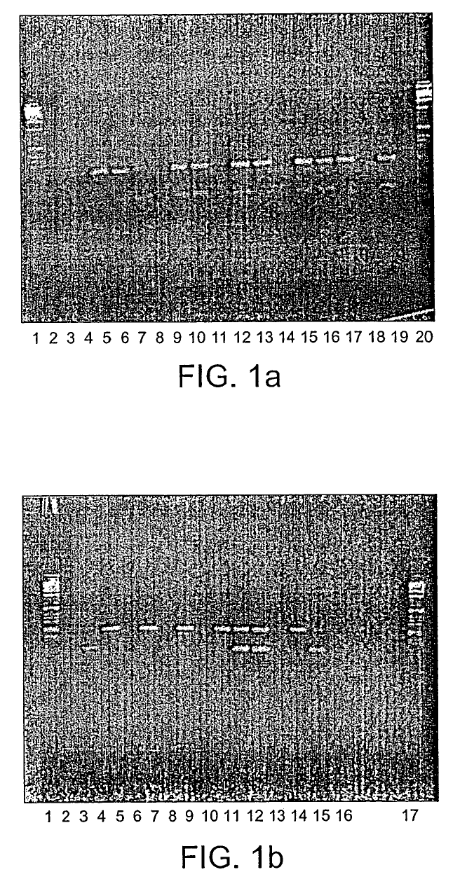

FIGS. 1a and 1b show 2% agarose gels of PCR products stained with ethidium bromide to show bands 88 base pairs in size (genotype 1 specific band) and 217 base pairs (all HCV genotypes).

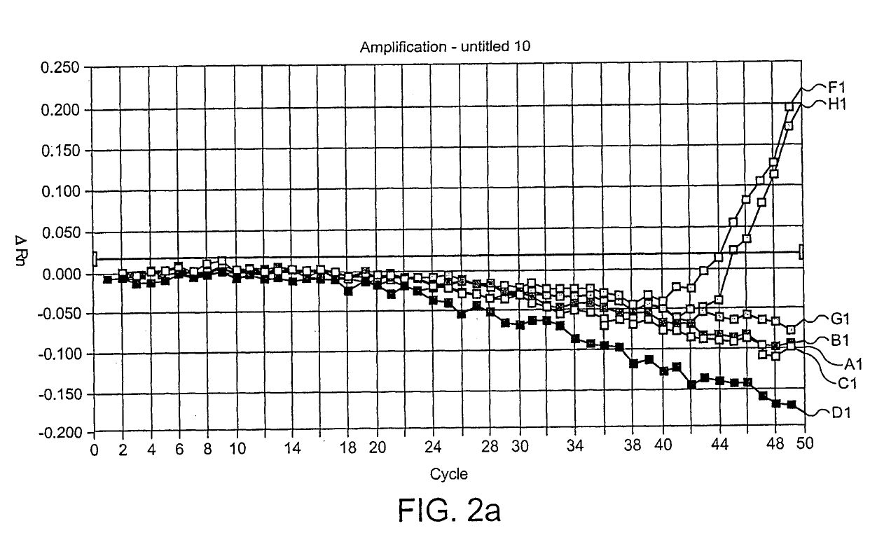

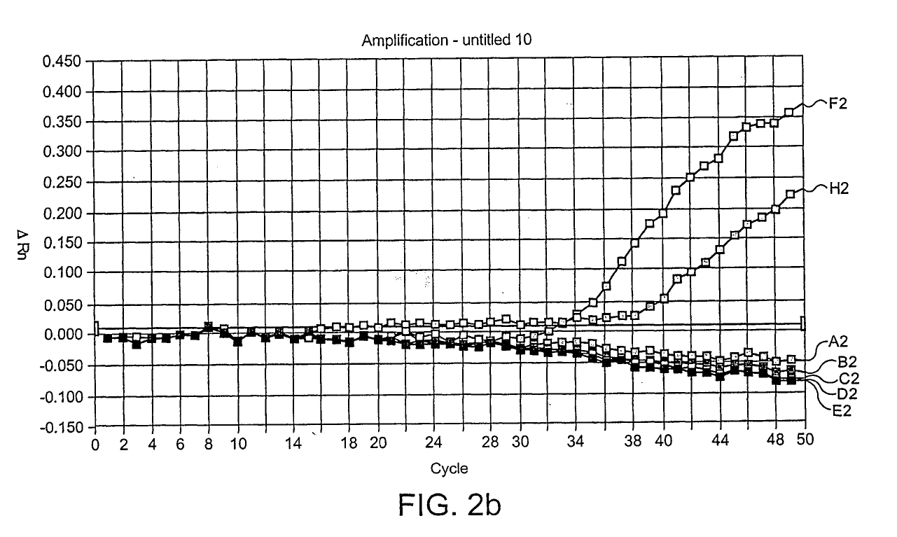

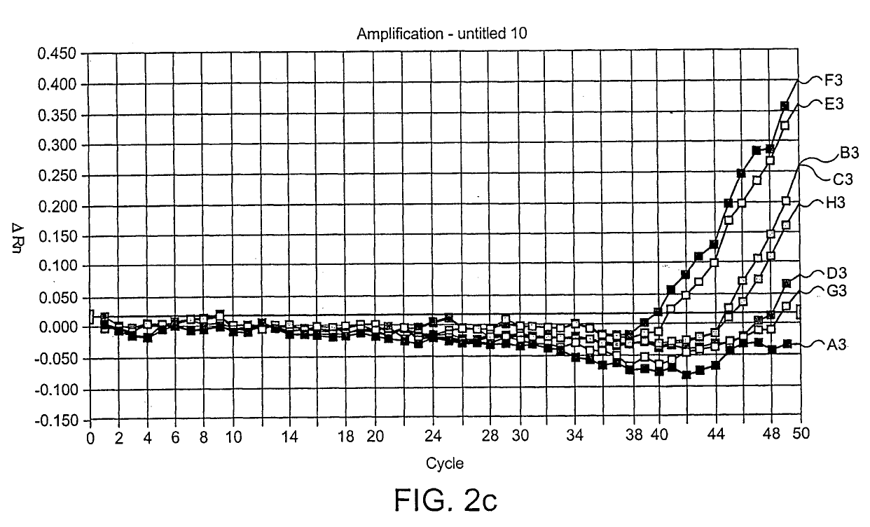

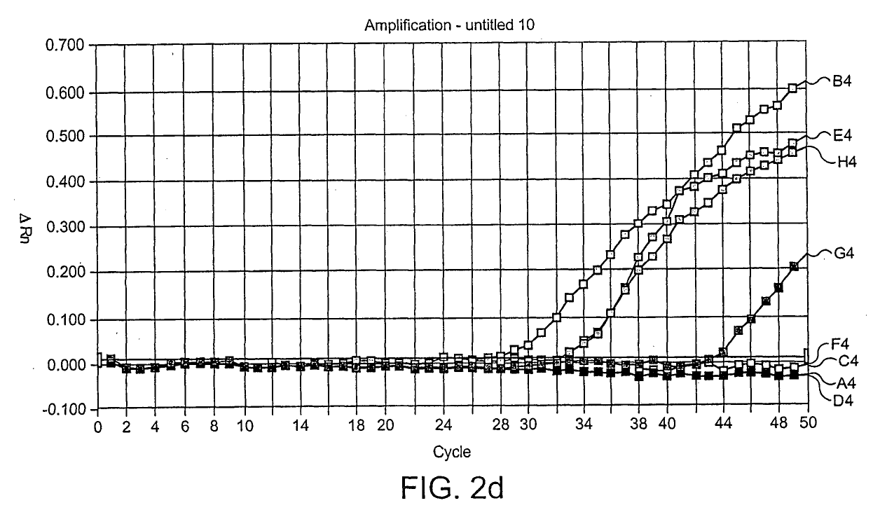

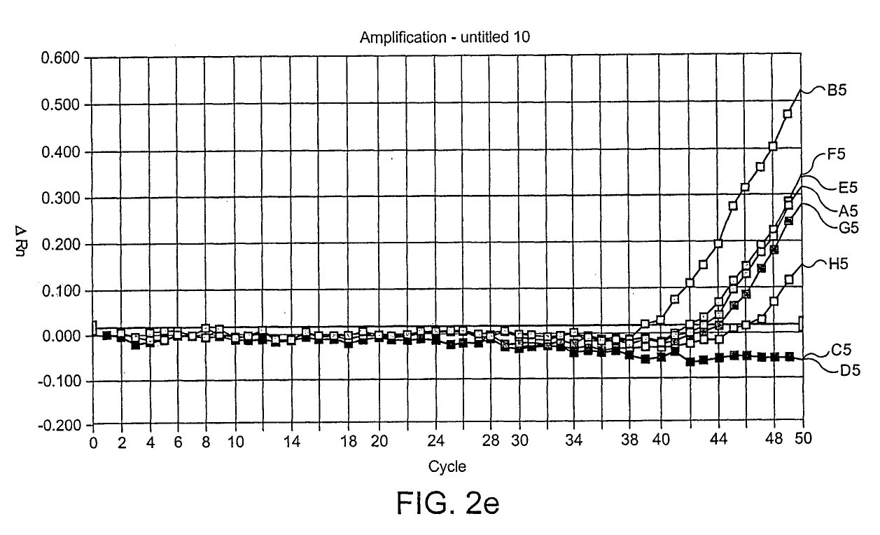

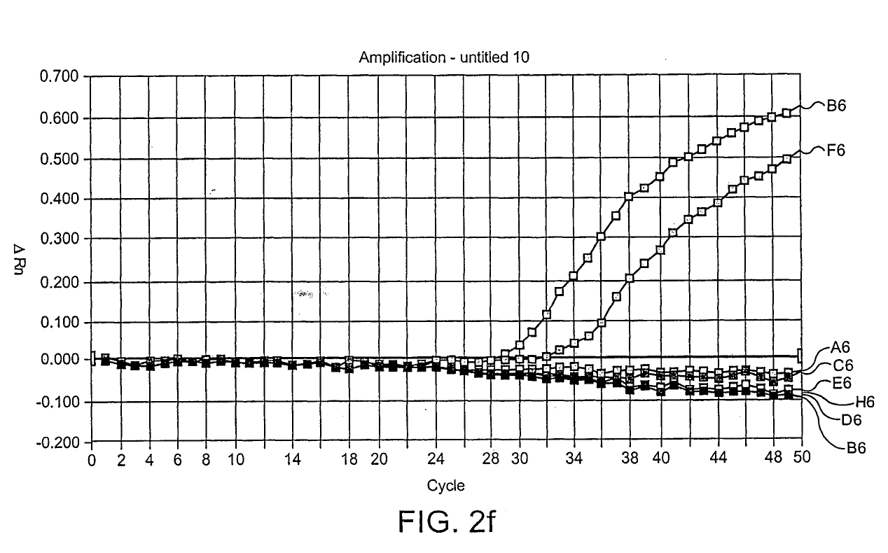

FIGS. 2a-f show printouts of PCR runs using fluorescent probe detection and Taqman technology. Fluorescence intensity is represented on the y axis and the number of PCR cycles elapsed is shown on the x axis.

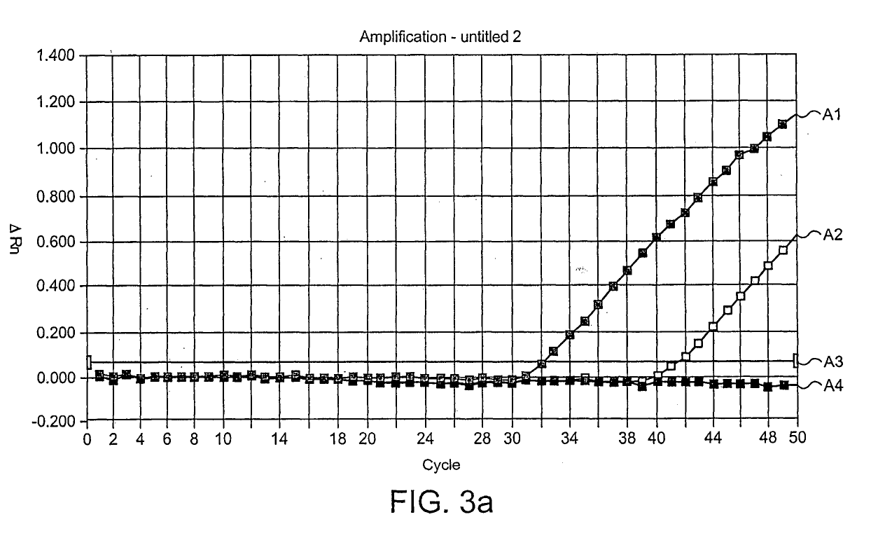

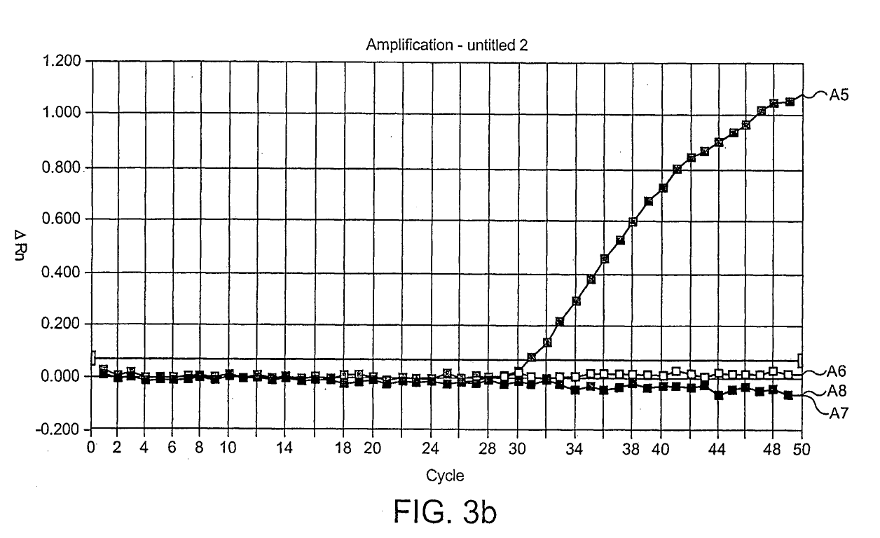

FIGS. 3a and 3b are printouts of PCR runs using fluorescent probe detection and Taqman technology. Fluorescence intensity is represented on the y axis and the number of PCR cycles elapsed is shown on the x axis.

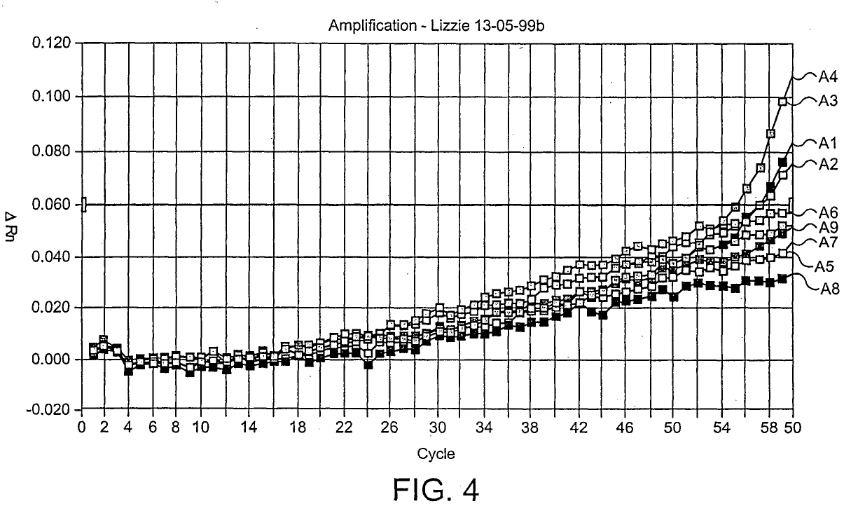

FIG. 4 shows printouts of PCR runs using molecular beacon probe analysis and fluorescence detection. Fluorescence intensity is represented on the y axis and the number of PCR cycles elapsed is shown on the x axis.

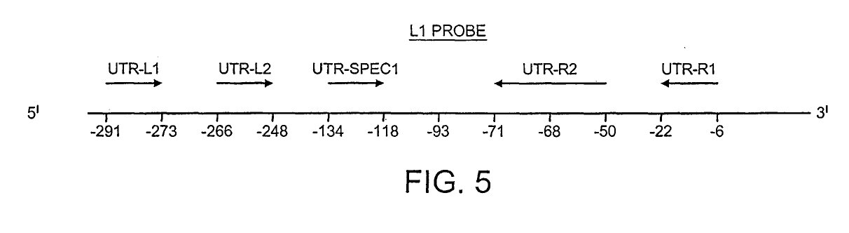

FIG. 5 is a diagram illustrating the positions of the various primers and probes used in certain embodiments of the present invention within the 5′NCR of the HCV-1 genome.

### EXAMPLES

### Example 1

From a 200 μl sample of serum, HCV RNA was extracted using mRNA extraction kit (QIAgen Ltd, Crawley, West Sussex) as per manufacturer's instructions. 11.5 μl of RNA solution as extracted above was added to 0.5 μl of random primers (Promega UK Ltd, Southampton) in a 1.5 μl eppindorf test tube and incubated at 90° C. for 5 minutes in a heating block and then snap cooled on ice.

cDNA was then prepared by reverse transcription using the following reaction mix:

5 μl of 5× SuperScript buffer (Life Technologies (GIBCO BRL) Paisley, Scotland); 2.5 μl of 100 mM DTT; 2.5 μl of 1 mg/ml Bovine Serum Albumin acetylated (10 mg/ml) (Promega, UK Ltd, Southampton); 1.25 μl of 10 mM dNTP solution (Amersham Pharmacia, Buckinghamshire); 1.25 μl Superscript Moloney Murine Leukaemia Virus reverse transcriptase (Life Technologies (GIBCO BRL) Paisley, Scotland); 0.5 μl of RNAsin ribonuclease inhibitor (Promega, UK Ltd, Southampton).

13 μl of this solution was added to each tube containing RNA/random primers. The mix was incubated at 42° C. for 60 minutes. The reaction was stopped by heating to 95° C. for 5 minutes. The cDNA was then stored at −20° C.

1 μl of the resulting complimentary DNA (cDNA) was suspended in 20 μl of a first round reaction mix. This mix contains 1.5 mM MgCl₂, 0.2 mM deoxy nucleotide triphosphates (guanine, adenosine, threonine and cytosine) (Amersham Pharmacia, Amersham, UK), 0.1 μl of Taq polymerase (Promega UK Ltd, Southampton) and 500 nM each of primers UTR-L1 and UTR-R1 (from Cuachem Ltd, Glasgow).

UTR-L1 is the forward primer and is complementary to residues −273 to −291 of the 5′UTR of HCV1. It has the sequence:

5′GGA ACT ACT GTC TTC ACG C3′

UTR-R1 is the reverse primer and is complementary to residues 6 to −22 of the 5′UTR of HCV1. It has the sequence:

5′ACG GTC TAC GAG ACC TC3′

The PCR reaction conditions were as follows: 94° C. for 1 minute for 1 cycle; 94° C. for 30 seconds; 55° C. for 20 seconds; 72° C. for 20 seconds for 25 cycles.

1 μl of this reaction was then added to 201 μl of a second round reaction mix. This mix is the same as the first round reaction mix, except that primers UTR-L1 and UTR-L2 are replaced by 500 mM of primers Spec-1 and UTR-R2, and 100 mM of UTR-L2.

UTR-L2 is a forward primer which is universal for all HCV genotypes and is complementary to residues −248 to −266 of the 5′UTR of HCV1. It has the following sequence:

5′CGT CTA GCC ATG GCG TTA G3′

Spec-1 is a forward primer which is HCV type 1 specific and is complementary to residues −118 to −134 of the 5′UTR of HCV1. It has the following sequence:

5′CCI CTC AAT GCC TGG AG3′

UTR-R2 is a reverse primer which is universal for all HCV genotypes and is complementary to residues −50 to −71 of the 5′UTR for HCV1. It has the following sequence:

5′GCA GTA CCA CAA GGC CTT TCG C3′

The reaction conditions were as follows: 94° C. for 1 minute for 1 cycle; 94° C. for 30 seconds; 58° C. for 20 seconds; 72° C. for 20 seconds for 25 cycles.

PCR products were then resolved by agarose gel electrophoresis (NuSieve 3:1, Flowgen Instruments, Lichfield, Staffordshire, UK) run for 40 minutes at 100 Volts in 2% agarose.

Samples of known HCV genotypes (determined by RFLP studies) were tested. The results are shown in FIGS. 1a and 1b. In FIG. 1a, the lanes contain (from left to right):
1—molecular weight markers
2—HCV negative control
3—HCV 3a
4—HCV 2b
5-HCV 1a
6—HCV 1a
7—negative control
8-HCV 1b
9—HCV 1b
10—water control
11-HCV 1b
12—HCV 2b
13—negative control
14—HCV 1a
15—HCV 1b
16-HCV3a
17—HCV negative control
18—1a positive control
19—water control
20—molecular weight markers

In FIG. 2a, the lanes contain (from left to right):
1—molecular weight markers
2—negative control
3—HCV 1a control
4—HCV 3a control
5—HCV negative control
6—HCV 5
7—negative control
8-HCV3a
9—negative control
10—HCV 2a
11-HCV 1b
12—HCV 1a
13—negative control
14—HCV 3a
15-HCV 1a
16—negative control
17—molecular weight markers

As can be seen, all HCV genotypes result in a 217 base pair band. Genotype 1 HCV generates an additional 88 base pair product.

### Example 2

RNA was isolated and cDNA produced in the same manner as in Example 1.

Two separate 25 μl reaction mixes containing 1 μl of cDNA, 3.5 mM MgCl₂, 0.2 mM each of dATP, dCTP, dGTP and 0.4 mM dUTP, 0.25 U of AmpliTaq Gold DNA Polymerase (Perkin Elmer), 300 nM of UTR-R2 and 300 nM of L-1 probe were prepared. One contained 300 nM of Spec-1 and the other contained 300 nM of UTR-L2.

L1 is a HCV genotype 1 specific probe (prepared by Scandinavian Gene Synthesis AB, Koping, Sweden) which is complementary to residues −68 to −93 of the 5′UTR of HCV1 and has the following sequence:

5′FCG CIA CCC AAC ICT ACT IGG CTA GT3′
where F = 6-FAM, 3′-T + TAMRA

The reaction conditions were as follows: 50° C. for 2 minutes, 95° C. for 10 minutes; for 1 cycle, 95° C. for 15 seconds, 60° C. for 60 seconds; for 60 cycles. Fluorescence was detected at 520 nm.

Samples of known HCV genotypes (determined by RFLP studies) were tested. The results are shown in FIGS. 2a-f. The following table provides a key to the conditions:

| | 1 | 2 | 3 | 4 | 5 | 6 |
|---|---|---|---|---|---|---|
| | (UTR-L2 + UTR- | (Spec-1 + UTR- | (UTR-L2 + UTR- | (Spec-1 + UTR- | (UTR-L2 + UTR- | (Spec-1 + UTR- |
| Primers | R2) | R2) | R2) | R2) | R2) | R2) |
| A | −ve control | −ve control | sample | Sample | 2a | 2a |
| B | −ve control | −ve control | 1a | 1a | 1b | 1b |
| C | −ve control | −ve control | 2b | 2b | Sample | Sample |
| D | −ve control | −ve control | 3a | 3a | sample | Sample |
| E | — | — | 1a | 1a | 3a | 3a |
| F | 1a | 1a | 5 | 5 | 1b | 1b |
| G | sample | sample | 1b | 1b | 3a | 3a |
| H | 1b | 1b | 1a | 1a | 2b | 2b |

Thus, it can be seen that HCV-1 can be differentiated from the other subtypes when the primer specific for HCV-1 is used (Spec-1) (see FIGS. 2b, d and f).

Similar results are seen in FIGS. 3a and 3b where the conditions are as follows:

| | 1 | 2 | 3 | 4 | 5 | 6 | 7 | 8 |
|---|---|---|---|---|---|---|---|---|
| | (UTR-L2 + UTR- | (UTR-L2 + UTR- | (UTR- | (UTR- | (SPEC-1 + UTR- | (SPEC-1 + UTR- | (SPEC-1 + UTR- | (SPEC-1 + UTR- |
| | R2) | R2) | L2) | L2) | R2) | R2) | R2) | R2) |
| A | 1a | 3a | −ve | −ve | 1a | 3a | −ve | −ve |
| | | | control | control | | | control | control |

This approach relies on sequence specific hybridisation of a fluorescently labelled probe to genotype 1 HCV sequences. These sequences are generated by PCR using at least one primer specific for HCV-1. The probe has a quencher molecule linked to the fluorochrome. As PCR proceeds, the quencher molecule is removed by Taq polymerase from incorporated probes. Fluorescence of the resulting genotype 1 PCR products can then be detected. The amount of fluorescence generated during the successive rounds of PCR correlates with the amount of PCR product.

### Example 3

RNA was isolated and cDNA produced in the same manner as in Example 1.

Two separate 25 μl reaction mixes containing 1 μl of cDNA, 3.5 mM MgCl₂, 0.2 mM each of dATP, dCTP, dGTP and 0.4 mM dUTP, 0.1 μl of Taq polymerase stoeffel fragment (Perkin Elmer), 0.1 μl of antiTaq antibody (Gibco BRL-Life Techonolgies, Paisley, Scotland), 60 nM carboxy-x-rhodamine (Cambridge Biosciences, Cambridge, UK), and 300 nM of UTR-R2-were prepared. One contained 300 nM of MBP-LR-ALL and the other contained 300 nM of MBP-LR-1 (Oswel DNA Service, Southampton).

MBP-LR-ALL is a forward primer which is universal for all HCV genotypes and is derived from the primer UTR-L2. It has the following sequence:

5′ FCA CCT TCA CCC TCA GAA GGM GCG UCT AGC CAT GGC
GTT AG 3′

MBP-LR-1 is a forward HCV type 1 specific primer which is derived from the primer Spec1. It has the following sequence:

5′ FCA CCT TCA CCC TCA GAA GGM GCC GCT CAA TGC CTG
GAG 3′

(F = FAM; M = MeREDdU and U = Uracil)

The results are shown in FIG. 4 where the conditions are as follows:

| | 1 | 2 | 3 | 4 | 5 | 6 | 7 | 8 | 9 |
|---|---|---|---|---|---|---|---|---|---|
| | (MBP-LR- | (MBP-LR- | (MBP-LR- | (MBP-LR- | (MBP-LR- | (MBP-LR- | (MBP- | (MBP-LR- | (MBP- |
| | 1 + UTR- | 1 + UTR- | 1 + UTR- | 1 + UTR- | 1 + UTR- | 1 + UTR- | LR-1 + UTR- | 1 + UTR- | LR-1 + UTR- |
| | R2) | R2) | R2) | R2) | R2) | R2) | R2) | R2) | R2) |
| A | 1a | 1a | 1a | 1a | 3a | 3a | −ve | −ve | −ve |
| | | | | | | | control | control | control |

1. A method of differentiating HCV genotype 1 (HCV-1) from HCV genotypes 2 and 3 (HCV-2 and HCV-3) in a sample, comprising:
subjecting the sample to an amplification reaction using at least one primer that anneals specifically to the 5′ noncoding region (5′ NCR) of the HCV-1 genome; and
detecting the product of the amplification reaction.

2. The method of claim 1, wherein the amplification reaction is the polymerase chain reaction (PCR) or reverse transcriptase polymerase chain reaction (RT-PCR).

3. The method of claim 1, further comprising:
detecting whether any HCV genotype is present in the sample by subjecting the sample to an amplification reaction using primers that anneal to a region of the 5′ NCR that is conserved between all HCV genotypes; and
detecting the product of this amplification reaction.

4. The method of claim 3, wherein the primers have the following sequences:
(UTR-L2, SEQ ID NO: 3)
Forward:
5′ CGT CTA GCC ATG GCG TTA G 3′
(UTR-R2, SEQ ID NO: 2)
Reverse:
5′ GCA GTA CCA CAA GGC CTT TCG C 3′.

5. The method of claim 1, further comprising subjecting the sample to a preliminary amplification reaction to isolate HCV material using primers universal for all HCV genotypes.

6. The method of claim 5, wherein the primers comprise the following sequences:
(UTR -L1, SEQ ID NO: 4)
Forward:
5′ GGA ACT ACT GTC TTC ACG C 3′
(UTR-R1, SEQ ID NO: 5)
Reverse:
5′ ACG GTC TAC GAG ACC TC 3′.

7. The method of claim 7, wherein the at least one primer which anneals specifically to the 5′ noncoding region (5′ NCR) of the HCV-1 genome is a forward primer and the reverse primer comprises the sequence:

| | |
|---|---|
| CCI CTC AAT GCC TGG AG 3′. | (Spec-1, SEQ ID NO: 1) |

8. The method of claim 7, wherein the at least one primer that anneals specifically to the 5′ noncoding region (5′ NCR) of the HCV-1 genome is a forward primer and the reverse primer comprises the sequence:
(UTR-R2, SEQ ID NO: 2)
5′ GCA GTA CCA CAA GGC CTT TCG C 3′.

9. The method of claim 1, wherein detection of the amplification reaction product is by agarose gel electrophoresis.

10. The method of claim 1, wherein detection of the amplification reaction product is by fluorescent analysis in which amplification of HCV-1 specific nucleic acid causes fluorescence of a probe.

11. The method of claim 10, wherein the probe comprises the sequence:
(L1, SEQ ID NO: 6)
5′ FCG CIA CCC AAC ICT ACT IGG CTA GT 3′
where F = 6-FAM, 3′-T + TAMRA.

12. The method of claim 1, wherein detection of the amplification reaction product is by one or more molecular beacon primers.

13. The method of claim 12, wherein the molecular beacon primer comprises the sequence:

| | | |
|---|---|---|
| 5′ FCA CCT TCA CCC TCA GAA | (MBP-LR-1,. | SEQ ID NO: 7) |
| GGM GCC GCT CAA TGC CTG | | |
| GAG 3′ | | |
| (F = FAM; M = MeREDdU and | | |
| U = Uracil) | | |

14. The method of claim 13, wherein the molecular beacon primer is a forward primer and the reverse primer comprises the sequence:

| | | |
|---|---|---|
| 5′ GCA GTA CCA CAA GGC CTT | (UTR-R2,. | SEQ ID NO: 2) |
| TCG C 3′ | | |

15. The method of claim 2, wherein the primers that anneal to a region of the 5′ NCR that is conserved between all HCV genotypes comprise the following sequences:

| | | | |
|---|---|---|---|
| Forward: | 5′ FCA CCT TCA CCC | MBP-LR- | SEQ ID NO: 8 |
| | TCA GAA GGM GCG UCT | ALL, | |
| | AGC CAT GGC GTT AG | | |
| | 3′ (F = FAM; M = | | |
| | MeREDdU and U = | | |
| | Uracil) | | |
| Reverse: | 5′ GCA GTA CCA CAA | (UTR-R2,. | SEQ ID NO: 2) |
| | GGC CTT TCG C 3′ | | |

16. A kit for detecting HCV genotype 1 (HCV-1) in a sample, comprising at least one primer that anneals specifically to the 5′ noncoding region (5′ NCR) of the HCV-1 genome.

17. (canceled)

18. A nucleotide suitable for use in an amplification reaction comprising one of the following sequences:

| | | |
|---|---|---|
| 5′ CGT CTA GCC CTG GCG | (UTR-L2, | SEQ ID NO: 3) |
| TTA G 3′ | | |
| 5′ GCA GTA CCA CAA CAA | (UTR-R2, | SEQ ID NO: 2) |
| GGC CTT TCG C 3′ | | |
| 5′ GGA ACT ACT GTC TTC | (UTR-L1, | SEQ ID NO: 4) |
| ACG C 3′ | | |
| 5′ ACG GTC TAC GAG ACC | (UTR-R1, | SEQ ID NO: 5) |
| TC 3′ | | |
| 5′ CCI CTC AAT GCC TGG | (Spec-1, | SEQ ID NO: 1) |
| AG 3′ | | |
| 5′ FCA CCT TCA CCC TCA | | |
| GAA GGM GCC GCT CAA TGC | | |
| CTG GAG 3′ | | |
| (F = FAM; M = MeREDdU | (MBP-LR-1, | SEQ ID NO: 7) |
| AND U = Uracil) | | |
| 5′ FCA CCT TCA CCC TCA | | |
| GAA GGM GCG UCT AGC CAT | | |
| GGC GTT AG 3′ | | |
| (F = FAM; M = MeREDdU | MBP-LR-ALL,. | SEQ ID NO: 8 |
| AND U = Uracil) | | |

19. A pair of primers comprising a nucleotide molecules having the following sequence:

| | | | |
|---|---|---|---|
| For- | 5′ CGT CTA GCC ATG | (UTR-L2, | SEQ ID NO: 3) |
| ward: | GCG TTA G 3′ | | |
| Re- | 5′ GCA GTA CCA CAA | (UTR-R2, | SEQ ID NO: 2) |
| verse: | GGC CTT TCG C 3′ | | |
| For- | 5′ GGA ACT ACT GTC | (UTR-L1, | SEQ ID NO: 4) |
| ward: | TTC ACG C 3′ | | |
| Re- | 5′ ACG GTC TAC GAG | (UTR-R1, | SEQ ID NO: 5) |
| verse: | ACC TC 3′ | | |
| For- | 5′ CCI CTC AAT GCC | (Spec-1, | SEQ ID NO: 1) |
| ward: | TGG AG 3′ | | |
| Re- | 5′ GCA GTA CCA CAA | (UTR-R2, | SEQ ID NO: 2) |
| verse: | GGC CTT TCG C 3′ | | |
| For- | 5′ FCA CCT TCA CCC | (MBP-LR-1, | SEQ ID NO: 7) |
| ward: | TCA GAA GGM GCC | | |
| | GCT CAA TGC CTG | | |
| | GAG 3′ (F = FAM; | | |
| | M = MeREDdU AND | | |
| | U = Uracil) | | |
| Re- | 5′ GCA GTA CCA CAA | (UTR-R2, | SEQ ID NO: 2) |
| verse: | GGC CTT TCG C 3′ | | |
| For- | 5′ FCA CCT TCA CCC | MBP-LR-ALL, | SEQ ID NO: 8 |
| ward: | TCA GAA GGM GCG | | |
| | UCT AGC CAT GGC | | |
| | GTT AG 3′ (F = | | |
| | FAM; M = MeREDdU | | |
| | AND U = Uracil) | | |
| Re- | 5′ GCA GTA CCA CAA | (UTR-R2,. | SEQ ID NO: 2) |
| verse: | GGC CTT TCG C 3′ | | |

20. A nucleotide molecule suitable for use as a probe comprising the sequence:

| | | |
|---|---|---|
| 5′ FCG CIA CCC AAC ICT ACT IGG | (L1, | SEQ ID NO: 6) |
| CTA GT 3′ | | |

(where F = 6-FAM, 3′-T + TAMRA).

21. A method of differentiating HCV genotype 1 (HCV-1) from HCV genotypes 2 and 3 (HCV-2 and HCV-3) in a sample, comprising:
subjecting the sample to an amplification reaction using at least one primer that anneals to the genome of HCV, a polymerase having a 5′-3′ exonuclease activity and an oligonucleotide probe, wherein the probe anneals specifically to the 5′ noncoding region (5′ NCR) of the HCV-1 genome and wherein the probe incorporates a modified nucleotide having a fluorescent characteristic that is modified by one or more neighboring nucleotides; and
detecting a change in fluorescence as the oligonucleotide probe is degraded by the exonuclease activity of the polymerase as the polymerase extends the primer and modification of the fluorescent characteristic of the modified nucleotide is reduced.

22. The kit of claim 16, wherein the primer allows for amplification by the polymerase chain reaction (PCR) or reverse transcriptase polymerase chain reaction (RT-PCR).

23. The kit of claim 16, further comprising a second primer, wherein the primer pair allow for an amplification reaction that a region of the 5′ NCR that is conserved between all HCV genotypes.

24. The kit of claim 23, wherein the primers have the following sequences:

| | | | |
|---|---|---|---|
| Forward: | 5′ CGT CTA GCC ATG | (UTR-L2, | SEQ ID NO: 3) |
| | GCG TTA G 3′ | | |
| Reverse: | 5′ GCA GTA CCA CAA | (UTR-R2,. | SEQ ID NO: 2) |
| | GGC CTT TCG C 3′ | | |

25. The kit of claim 16, further comprising reagents and primers suitable a preliminary amplification reaction to isolate HCV material using primers universal for all HCV genotypes.

26. The kit of claim 25, wherein the primers comprise the following sequences:

| | | | |
|---|---|---|---|
| Forward: | 5′ GGA ACT ACT GTC | (UTR-L1, | SEQ ID NO: 4) |
| | TTC ACG C 3′ | | |
| Reverse: | 5′ ACG GTC TAC GAG | (UTR-R1,. | SEQ ID NO: 5) |
| | ACC TC 3′ | | |

27. The kit of claim 23, wherein the forward primer for the amplification reaction anneals specifically to the 5′ noncoding region (5′ NCR) of the HCV-1 genome and the reverse primer comprises the sequence:

| | | |
|---|---|---|
| CCI CTC AAT GCC TGG AG 3′ | (Spec-1,. | SEQ ID NO: 1) |

28. The kit of claim 23, wherein the forward primer for the amplification reaction anneals specifically to the 5′ noncoding region (5′ NCR) of the HCV-1 genome and the reverse primer comprises the sequence:

| | | |
|---|---|---|
| 5′ GCA GTA CCA CAA GGC CTT | (UTR-R2,. | SEQ ID NO: 2) |
| TCG C 3′ | | |

29. The kit of claim 23, further comprising reagents sufficient for detection of the amplification products by fluorescent analysis in which amplification of HCV-1 specific nucleic acid causes fluorescence of a probe.

30. The kit of claim 29, wherein the probe comprises the sequence:
(L1, SEQ ID NO:6)
5′ FCG CIA CCC AAC ICT ACT IGG CTA GT 3′
where F = 6-FAM, 3′-T + TAMRA.
